(19) United States
(12) Patent Application Publication
(10) Pub. No.: US 2006/0076010 A1
(43) Pub. Date: Apr. 13, 2006

(54) Drug delivery system with vented mouthpiece

(76) Inventor: Michael L. King, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/537,085

(22) PCT Filed: Dec. 4, 2003
(86) PCT No.: PCT/US03/38483

§ 371 Date: Jun. 1, 2005

Related U.S. Application Data
(60) Provisional application No. 60/434,517, filed on Dec. 18, 2002.

(51) Int. Cl. A61M 11/00; A61M 15/00

(52) U.S. Cl. 128/200.23; 128/203.15

(57) ABSTRACT

An oral inhaler suitable for delivering a pharmaceutical formulation to a patient that includes a container having the pharmaceutical formulation including at least one medicament present therein, and a mouthpiece configured for oral engagement with a patient and in communication with the container, wherein the mouthpiece has an inner surface and an outer surface, is described. The outer surface of the mouthpiece contains at least one longitudinally-extending disuniformity such that when the patient orally engages the mouthpiece at least one void space is created between the outer surface of the mouthpiece and the oral cavity of the patient so as to provide an air flow channel through the at least one void space to facilitate intake of the at least one medicament by the patient.

### CROSS-REFERENCE TO RELATED APPLICATIONS

The present application claims priority to Provisional Application No. 60/434,517 filed Dec. 18, 2002, the disclosure of which is incorporated herein by reference in its entirety.

### FIELD OF THE INVENTION

The present invention generally relates to systems for delivering medicaments to patients and methods of using the same.

### BACKGROUND OF THE INVENTION

A variety of systems for orally delivering medicaments in a fluid medium are widely known in the art. Examples include oral inhalers such as aerosol systems which typically deliver one or more medicaments in combination with a propellant (e.g., metered dose inhalers commonly known as MDIs), as well as systems that utilize dry powder formulations (e.g. dry powder inhalers commonly known as DPIs)

With respect to oral inhalers, medicaments, broadly including therapeutic, prophylactic and diagnostic agents, may be delivered locally to the lung or systemically through the lung for the treatment, prophylaxis or diagnosis of illnesses and other conditions. As an example, MDIs are aerosol delivery systems having a reservoir of compressed, low boiling point liquid propellant formulated with a medicament. Inhalers are designed to deliver a metered dose of the medicament formulation, dispensing the dose as an inhalable particulate cloud, or plume.

To deliver the medicament dose to the patient, inhalers typically have an interface with the patient. This interface is usually in the form of a mouthpiece which the patient inserts into the mouth. Many inhalers, especially those intended for drug delivery to the lungs, often require the patient to inhale during the delivery of the dose. When used as directed, conventional oral inhalers usually result in a substantially sealed interface around the perimeter of the mouthpiece. Thus, airflow generated by patient inhalation, for the most part, comes through (at least some portion of) the device itself. In light of conventional inhaler configurations, it may be desirable to completely, substantially, or partially restrict airflow through the device. For example, it may be desirable to close off the region internal to the mouthpiece near its base in order to enclose the internal components of the inhaler. As another example, it may be desirable to eliminate airflow inlets on the outer surfaces of the body of the inhaler to prevent them being covered by the patient's hand during use, or to protect the internal components of the inhaler. Thus, there is a need for an inhaler mouthpiece that allows a patient to inhale freely during dose delivery while not requiring an airflow path through the inhaler.

In one instance, the patient holds the MDI at a predetermined distance from the mouth (e.g., several inches) and fires the inhaler. As a result, little if any airflow goes through the device and all airflow comes from immediately around the mouth. This “open mouth technique” is subject to poor orientation and aim of the inhaler, as well as increased patient-to-patient variation. Accordingly, such a technique is disadvantageous. Examples of references to the open mouth technique are as follows:

http://www.aaaai.org/patients/publicdmat/tips/inhaledmedications.stm

http://www.asthma-education.com/howto0.1.html

http://www.uhn.ca/programs/asthma/Pages/inhalecare.html

http://www.starbright.org/schoolasthma/pdf/mdi white paper.pdf

http://www.keepkidshelathy.com/asthma/using a mdi.html

http://shs.unc.edu/medservices/specialty services/asthma/inhaler.html

http://www.asthmacentre.com/manual/openmdi.html

Airflow through an inhalation device may have the potential to influence the plume and thus the deposition profile of the dose of medicament. Airflow in the dose delivery passage of an inhaler, intentionally or unintentionally, typically interacts with the plume during dose delivery. A portion of the medicament particles from each dose usually deposits on the target (e.g., lungs, nasal passages, etc), a portion on the inhalation device, and a portion on a non-targeted area of the patient (e.g., oropharynx, etc). It is generally desirable to maximize the amount of each dose that deposits on the intended target, while minimizing the unwanted deposition on the device and non-targeted areas of the patient. Thus, it may be desirable to configure the airflow path to reduce unwanted deposition.

One attempt to address this problem is proposed by WO 00/50112. In particular, WO 00/50112 relates to a pressurized metered dose inhaler having an actuator constructed and arranged so as to inhibit airflow due to patient inhalation in the vicinity of the orifice of the nozzle block when the valve stem is in the dispensing position. WO 00/50112 discloses that such a design reduces unwanted oropharyngeal deposition of medicament and increases the relative amount of medicament to the lung. Notwithstanding any possible advantages related to the teachings of WO 00/50112, such a design is believed to require an additional structural component to be employed with the inhaler.

There remains a need in the art to provide alternative airflow configurations by modifying the medicament delivery device in a relatively less complicated manner than currently realized in the art. There is a need for such configurations for use in a wide number of oral inhalers including, without limitation, MDIs and DPIs.

### SUMMARY OF THE INVENTION

In one aspect, the invention provides an oral inhaler suitable for delivering a pharmaceutical formulation to a patient. The inhaler comprises a container having the pharmaceutical formulation comprising at least one medicament present therein; and a mouthpiece configured for oral engagement with a patient and in communication with the container, with the mouthpiece having an inner surface and an outer surface. Advantageously, the outer surface of the mouthpiece contains at least one longitudinally-extending disuniformity such that when the patient engages the mouthpiece at least one void space is created between the outer surface of the mouthpiece and the patient so as to provide an air flow channel through the at least one void space to facilitate intake of the at least one medicament by the patient. Accordingly, the patient is allowed to inhale freely during dose delivery without an airflow path internal to the inhalation device being required.

In another aspect, the invention provides a method of administering at least one medicament to a patient. The method comprises providing an oral inhaler as defined herein; and activating the oral inhaler to deliver the at least one medicament to the patient.

These and other aspects and advantages of the invention are set forth herein.

### BRIEF DESCRIPTION OF THE DRAWINGS

FIGS. 1A through 1D respectively illustrate perspective, side cross-sectional, top, and frontal views of an oral inhaler according to the present invention.

FIGS. 2A through 2E respectively illustrate perspective, side cross-sectional, top, frontal and bottom cross-sectional views of a conventional oral inhaler.

FIGS. 3A through 3D respectively illustrate perspective, side cross-sectional, top, and frontal views of an oral inhaler according to the present invention.

FIGS. 4A through 4E respectively illustrate perspective, side cross-sectional, top, frontal and bottom cross-sectional views of an oral inhaler according to the present invention.

FIG. 5 illustrates a perspective view of an oral inhaler according to the present invention.

FIG. 6 illustrates a perspective view of a dry powder inhaler in accordance with the present invention.

FIG. 7 illustrates a side view of a patient utilizing an oral inhaler in accordance with the present invention.

### DETAILED DESCRIPTION OF THE INVENTION

The invention will now be described with respect to the embodiments set forth herein, which include, without limitation, the accompanying drawings. It should be appreciated that these embodiments are merely set forth to illustrate the invention, and are not to be construed as limiting the scope of the invention.

All publications, patents, and patent applications cited herein, whether supra or infra, are hereby incorporated herein by reference in their entirety to the same extent as if each individual publication, patent, or patent application was specifically and individually indicated to be incorporated by reference.

It must be noted that, as used in the specification and appended claims, the singular forms “a”, “an” and “the” include plural referents unless the content clearly dictates otherwise.

In one aspect, the invention provides an oral inhaler suitable for delivering a pharmaceutical formulation to a patient. The inhaler comprises a container having the pharmaceutical formulation comprising at least one medicament present therein; and a mouthpiece configured for oral engagement with a patient and in communication with the container, with the mouthpiece having an inner surface and an outer surface. Advantageously, the outer surface of the mouthpiece contains at least one longitudinally-extending disuniformity such that when the patient engages the mouthpiece at least one void space is created between the outer surface of the mouthpiece and the patient so as to provide an air flow channel through the at least one void space to facilitate intake of the at least one medicament by the patient. Accordingly, the patient is allowed to inhale freely during dose delivery without an airflow path internal to the inhalation device or an additional mouthpiece component being required.

For the purposes of the invention, and as set forth in greater detail herein, the system may encompass a wide variety of inhalers including, without limitation, metered dose inhalers (MDIs) and dry powder inhalers (DPIs). Examples of such inhalers and inhaler components are described in commonly assigned U.S. Pat. Nos. 4,364,923; 6,309,624; 4,335,121; 6,251,368; 5,676,929; 5,674,471; 5,290,815; 5,126,375; 5,225,445; 4,922,474; 5,674,472; 5,658,549; 5,270,305; 6,303,103; 6,309,624; 6,315,173; 6,170,717; 6,318,603; 6,238,647; 6,119,853; 6,315,112; 6,179,118; 6,149,892; 6,253,762; 6,131,566; 6,143,277, 5,590,645; 5,860,419; 5,873,360; 6,032,666; and 6,378,519, and U.S. patent application Ser. Nos. 09/925,214 and 10/022,072, the disclosures of which are all incorporated herein by reference.

For the purposes of the invention, the term “longitudinally-extending “disuniformity” refers to, for example, a variation, modification, inconsistency, vent, or disruption in the outer surface of the mouthpiece which extends along the length of the mouthpiece. . The configuration of the “disuniformity” varies depending on the design of the inhaler or mouthpiece. In a preferred embodiment, multiple disuniformities are distributed symmetrically about the axis of the mouthpiece. In accordance with the invention, the presence of the disuniformity provides a void space between the contact surfaces of the oral cavity of a patient and the outer surface of the mouthpiece so as to provide an air flow channel therethrough.

The disuniformity may be present in a variety of forms. Such forms include, without limitation, a protrusion, an indentation, or an opening in the outer surface. Combinations of such disuniformities may also be employed.

For the purposes of the invention, the term “protrusion” refers to a projection, such as for example a rib, that extends outward from the outer surface of the mouthpiece. The term “indentation” refers to a recess in the external surface of the mouthpiece. The term “opening” refers to the surface of the mouthpiece being unrestricted such that the environment external to the medicament delivery system is directly exposed to the internal void defined by the inner surface of the mouthpiece. It should be appreciated that a single protrusion, indentation, or opening can be employed, or a plurality of protrusions, indentations, and/or openings.

The disuniformity can extend at various lengths along the longitudinal axis of the mouthpiece, which may be selected as deemed appropriate. For example, in one embodiment, the disuniformity may extend throughout the length of the mouthpiece. Other shorter lengths are also contemplated, i.e., the disuniformity may extend only throughout a portion of the longitudinal axis.

In the event that a plurality of disuniformities are employed, the distance between them can vary as desired. As an example, in one embodiment, the disuniformities may be equidistant from each other. Conversely, distances between individual disuniformities can also be unequal.

The disuniformity may be present at any number of locations along the outer surface area of the mouthpiece, which encompasses the top, bottom and opposing sides of the mouthpiece. As an example, in one embodiment, in the event that a plurality of disuniformities are employed, a predetermined number of such disuniformities can be present opposite to each other, such as being present on the top and bottom, or on the two sides. In a particular embodiment, an equal number of disuniformities may be present on opposing sides of the mouthpiece. Moreover, the disuniformities can be present on adjacent outer surface portions, for example, top and side in one embodiment or bottom and side in another embodiment. Furthermore, in another embodiment, disuniformities can be present throughout the outer surface of the mouthpiece, i.e., on the top, bottom, and sides.

The term “container” refers to various receptacles for holding the pharmaceutical formulation. Examples of such containers include, without limitation” canisters capable of withstanding pressure such as those that are employed in conjunction with aerosol formulations for MDIs. With respect to DPIs, examples of containers include, without limitation, pockets or pocket-like structures, such as those typically present in peelable blister packages used in conjunction with dry powder pharmaceutical formulations. In general, containers may be formed from materials known to one in the art including, without limitation, polymers, metal, and glass, as well as others.

Other accessories may be used in conjuction with the devices described herein without departing from the scope of the present invention. Such accessories include, for example, spacers.

Medicaments, which may be administered in the formulations, include, without limitation, various drugs useful in inhalation therapy. Appropriate medicaments may thus be selected from, for example, analgesics, (e.g., codeine, dihydromorphine, ergotamine, fentanyl or morphine); anginal preparations, (e.g., diltiazem; antiallergics, e.g., cromoglycate, ketotifen or nedocromil); antiinfectives (e.g., cephalosporins, penicillins, streptomycin, sulphonamides, tetracyclines and pentamidine); antihistamines, (e.g., methapyrilene); anti-inflammatories, (e.g., beclomethasone dipropionate, fluticasone propionate, flunisolide, budesonide, rofleponide, mometasone furoate, ciclesonide, triamcinolone acetonide or 6α, 9α-difluoro-11β-hydroxy-16α-methyl-3-oxo-17α-propionyloxy-androsta-1,4-diene-17β-carbothioic acid S-(2-oxo-tetrahydro-furan-3-yl) ester)); antitussives, (e.g., noscapine; bronchodilators, e.g., albuterol (e.g. as sulphate), salmeterol (e.g. as xinafoate), ephedrine, adrenaline, fenoterol (e.g as hydrobromide), formoterol (e.g., as fumarate), isoprenaline, metaproterenol, phenylephrine, phenylpropanolamine, pirbuterol (e.g., as acetate), reproterol (e.g., as hydrochloride), rimiterol, terbutaline (e.g., as sulphate), isoetharine, tulobuterol or 4-hydroxy-7-[2-[2-[[3-(2-phenylethoxy)propyl]sulfonyl]ethyl]amino]ethyl-2(3H)-benzothiazolone); diuretics, (e.g., amiloride; anticholinergics, e.g., ipratropium (e.g., as bromide), tiotropium, atropine or oxitropium); hormones, (e.g., cortisone, hydrocortisone or prednisolone); xanthines, (e.g., aminophylline, choline theophyllinate, lysine theophyllinate or theophylline); therapeutic proteins and peptides, (e.g., insulin). It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament. It will be further clear to a person skilled in the art that where appropriate, the medicaments may be used in the form of a pure isomer, for example, R-salbutamol or RR-formoterol.

Particularly preferred medicaments for administration using pharmaceutical formulations in accordance with the invention include anti-allergics, bronchodilators and anti-inflammatory steroids of use in the treatment of respiratory disorders such as asthma by inhalation therapy, for example cromoglycate (e.g. as the sodium salt), salbutamol (e.g. as the free base or the sulphate salt), salmeterol (e.g. as the xinafoate salt), formoterol (e.g. as the fumarate salt), terbutaline (e.g. as the sulphate salt), reproterol (e.g. as the hydrochloride salt), a beclomethasone ester (e.g. the dipropionate), a fluticasone ester (e.g. the propionate). Medicaments useful in erectile dysfunction treatment (e.g., PDE-V inhibitors such as those employed in Vardenafil®) of GlaxoSmithKline located in Research Triangle Park, N.C., along with alprostadil and sildenafil citrate) may also be employed.

Salmeterol, especially salmeterol xinafoate, salbutamol, especially salbutamol sulphate, fluticasone propionate, beclomethasone dipropionate and physiologically acceptable salts and solvates thereof are especially preferred.

It will be appreciated by those skilled in the art that the formulations according to the invention may, if desired, contain a combination of two or more active ingredients. Compositions containing two active ingredients are known for the treatment of respiratory disorders such as asthma, for example, formoterol (e.g. as the fumarate) and budesonide, salmeterol (e.g. as the xinafoate salt) and fluticasone (e.g. as the propionate ester), salbutamol (e.g. as free base or sulphate salt) and beclomethasone (as the dipropionate ester) are preferred.

A particularly preferred combination is a combination of fluticasone propionate and salmeterol, or a salt thereof (particularly the xinafoate salt). It should be understood that the medicaments that may be used in conjunction with the delivery system are not limited to those described herein.

The oral inhaler may be operated in various and accepted manners. For example, with respect to an MDI, in one embodiment, the canister is depressed into the actuator. The motion of the canister causes the metering valve to meter a fixed volume of the fluid forming an individual dose. The metered dose of the fluid passes into and through the valve stem, nozzle, and mouthpiece, i.e., mouthpiece. Upon leaving the pressurized environment of the canister and metering chamber, the propellant component of the fluid expands, carrying the dose to the user.

A DPI may be used by a patient orally engaging the DPI mouthpiece and inhaling air therethrough. When a patient inhales through the mouthpiece, air flows through a pocket containing a dose of pharmaceutical formulation , and eventually out through the mouthpiece, entraining the powder and thus carrying the dose to the patient.

Various embodiments of DPI components that may be employed in accordance with the invention is described in U.S. Pat. Nos. 5,590,645; 5,860,419; 5,873,360; 6,032,666; 6,378,519 and U.S. application Ser. Nos. 09/925,214 and 10/022,072. Such embodiments encompass DISKUS® inhalers made commercially available by GlaxoSmithKline of Research Triangle Park, N.C. More specifically, in such embodiments, DPI includes a medicament pack having containers (e.g., pockets) which hold dry powder pharmaceutical formulation therein spaced along the length of, and defined between, two peelable sheets secured to each other. The DPI includes: (1) an opening station for receiving a container of a medicament pack used within the device, (2) means positioned to engage peelable sheets of a container which has been received in the opening station for peeling apart the peelable sheets for opening the container, and (3) an outlet, positioned to be in communication with an opened container through which a user can inhale medicament in powder form from such an open container.

Other embodiments of a DPI that is encompassed by the present invention include those described in U.S. Pat. Nos. 4,627,432; 4,811,731; 5,035,237; 4,778,054; and Des 299,066. Such embodiments encompass ROTODISK® inhalers made commercially available by GlaxoSmithKline. In particular, such a device may include a housing, a tray mounted in the housing and movable between first and second positions relative to the housing, a support disk provided on the tray and adapted to receive a carrier provided with at least one medicament container. A plunger may be present which is operable to penetrate a container registered therewith to open the container.

In embodiments pertaining to MDIs, preferred formulations for use in the canisters of the present invention comprise at least one medicament and at least one propellant. For the purposes of the invention, the term ‘propellant’ means pharmacologically inert liquids with boiling points from about room temperature (25° C.) to about −25° C. which singly or in combination exert a high vapor pressure at room temperature including CFCs such as freon and hydrofluorcarbons. The propellants used in the present invention are low boiling fluorocarbons, in particular, hydrofluorocarbons or hydrofluoroalkanes. Examples of preferred propellants include, but are not limited to, a C₁₋₄ hydrofluoroalkane, e.g., 1,1,1,2-tetrafluoroethane and 1,1,1,2,3,3,3-n-heptafluoropropane, or a mixture thereof as propellant. Other propellants may be used including, for example, alkanes (e.g., butane and propane), along with CO₂ (e.g., liquid CO₂).

With respect to DPIs, dry powder pharmaceutical formulations administered therefrom may include, in addition to one or more medicaments, at least one ingredient. Such ingredients include, without limitation, excipients (e.g., lactose, sucrose, D-mannitol, starch, corn starch, crystalline cellulose, and light silicic anhydride), lubricants (e.g., magnesium stearate, calcium stearate, talc, and colloidal silica), binders (e.g., crystalline cellulose, sucrose, D-mannitol, dextrin, hydroxypropylcellulose, hydroxypropylmethylcellulose, polyvinylpyrrolidone, starch, gelatin, methylcellulose, and carboxymethylcellulose sodium), disintegrators (e.g., starch, carboxymethylcellulose, carboxymethylcellulose calcium, croscarmellose sodium, carboxymethylstarch sodium, and L-hydroxypropylcellulose), solvents (e.g., water, alcohol, propylene glycol, macrogols, sesame oil, corn oil, and olive oil), solubilizers (e.g., polyethylene glycol, propylene glycol, D-mannitol, benzyl benzoate, ethanol, trisaminomethane, cholesterol, triethanolamine, sodium carbonate, and sodium citrate). Co-solvents can also be employed and include, without limitation, C₁-C₄ alcohols (e.g., methanol, ethanol, isopropanol, butanol). Mixtures of any of the above ingredients may also be employed.

In another aspect, the invention provides a method of administering at least one medicament to a patient. The method comprises providing an oral inhaler as defined herein; and activating the oral inhaler to deliver the at least one medicament to the patient. Such activation may be carried out, for example, according to embodiments described herein, although it should be appreciated that other techniques can also be employed.

The invention will now be described with respect to the accompanying drawings. It should be appreciated that the drawings merely illustrate embodiments of the present invention, and do not serve to limit the scope of the invention as defined by the claims.

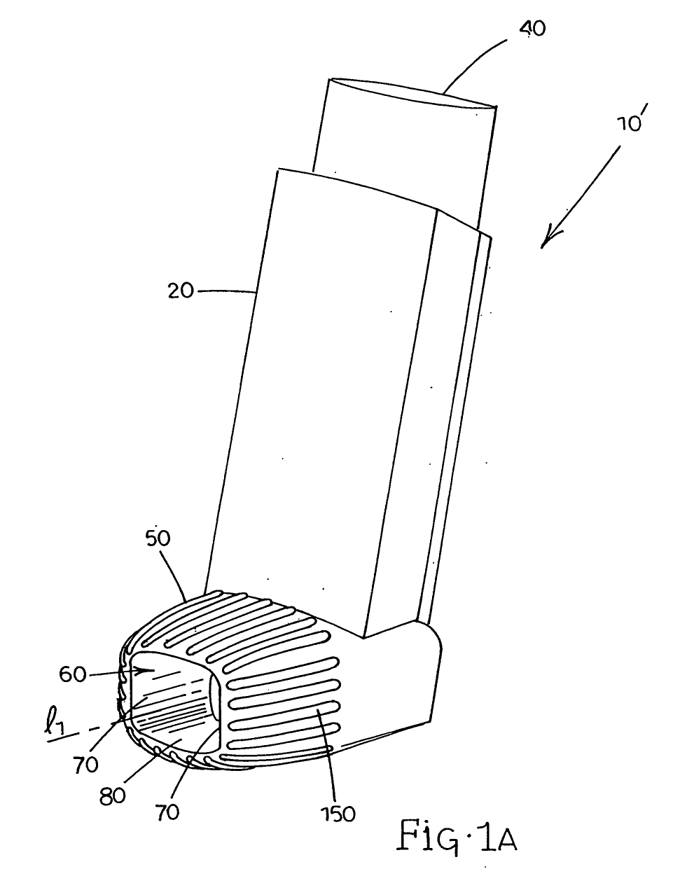

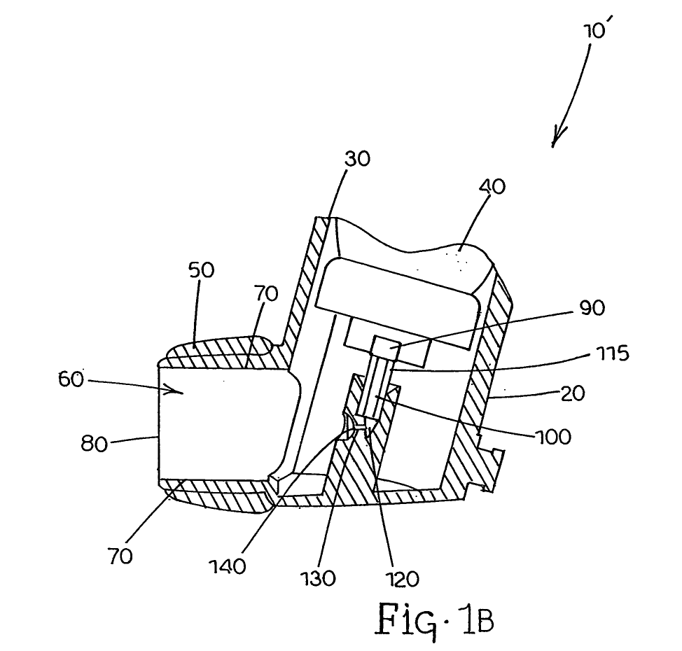

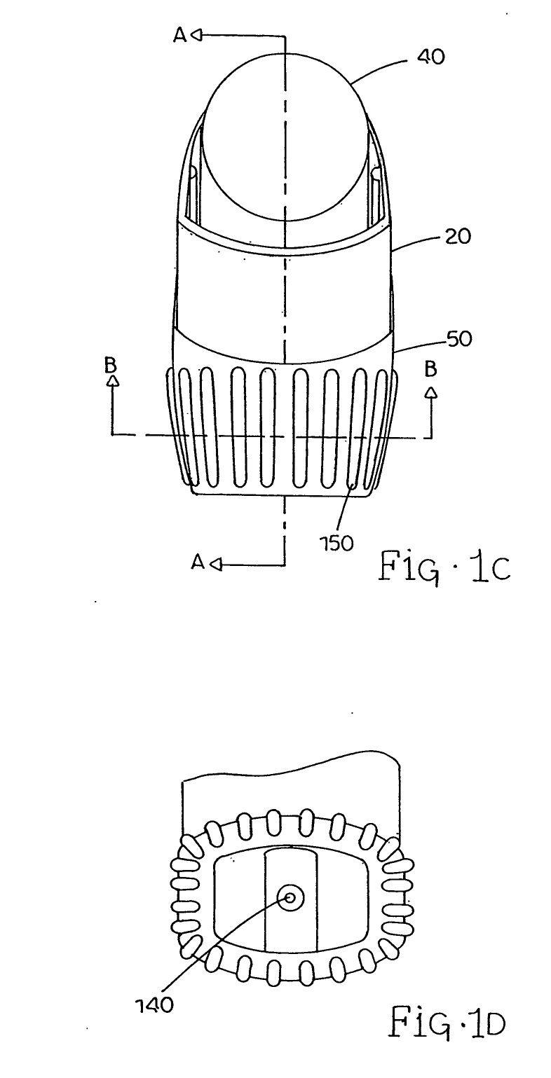

FIGS. 1A through 1D respectively illustrate perspective, side cross-sectional, top and frontal views of an embodiment of an oral inhaler 10′ in accordance with the present invention. The inhaler 10′ is preferably present as an MDI. As depicted, the inhaler 10′ includes a housing 20 with a cavity 30 formed therein adapted to receive a canister 40. The canister 40 contains a quantity of medicament in suspension or solution with a pressurized liquid propellant that is gaseous at room temperature. Also included is a mouthpiece 50 having a chamber 60 with interior walls 70, an open (distal) end 80, and either a rear wall or a second open end (not shown). The housing 20 and the mouthpiece 50 may be a unitary structure or may be of one-piece construction.

FIG. 1B depicts a cross-sectional view of inhaler 10′. As shown, canister 40 possesses a metering assembly 90 (e.g., valve) for metering a dose of pressurized liquid medicament. The metering assembly 90 is in communication with the mouthpiece 50. Also included is a valve stem 100 for releasing the metered dose in communication with the metering assembly 90. The housing further includes a nozzle block 110 containing a valve stem seat 115 for engaging the valve stem 100, an expansion chamber 120 in fluid communication with the valve stem 100, and a nozzle channel 130 in fluid communication with the expansion chamber 120. The nozzle channel 130 has an exit orifice or nozzle 140 at one end. As depicted in FIG. 1B, the nozzle 140 is aligned with the open end of the conduit. One embodiment of the metering valve, expansion chamber, and nozzle is set forth in FIG. 1B. However, it should be appreciated that numerous deviations from this embodiment can be made without departing from the scope of the invention.

As shown in the perspective view provided by FIG. 1A, a plurality of protrusions (e.g., ribs) 150 are present on the top, bottom and opposing sides of the outer surface of the inhaler mouthpiece 50. More specifically, and as depicted, the protrusions extend coaxially along longitudinal axis I₁ of the mouthpiece. The protrusions 150 may be equidistant from each other, or alternatively may be spaced apart at different distances. In either instance, gaps, i.e., void spaces, that are present between the protrusions serve as air flow channels to facilitate intake of medicament by a patient.

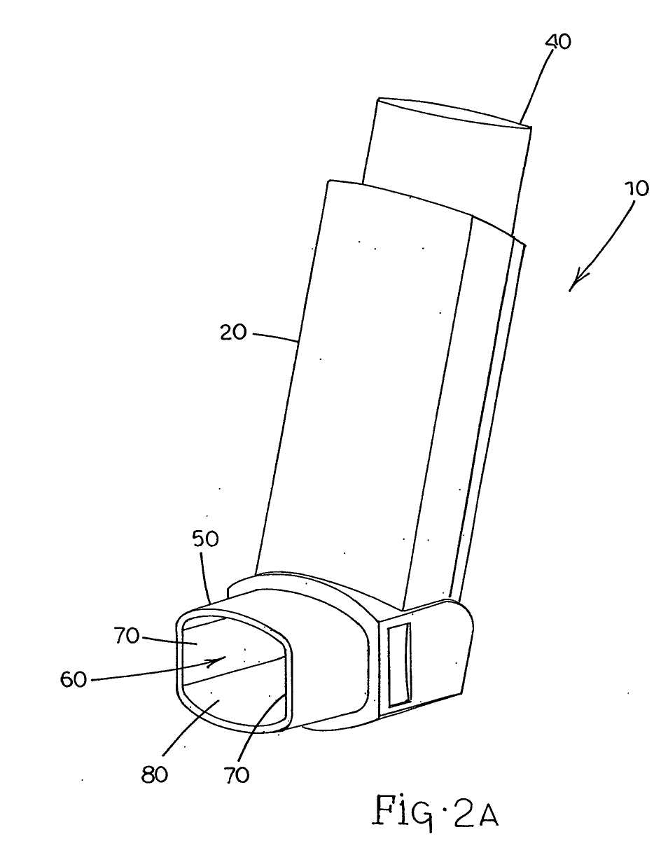

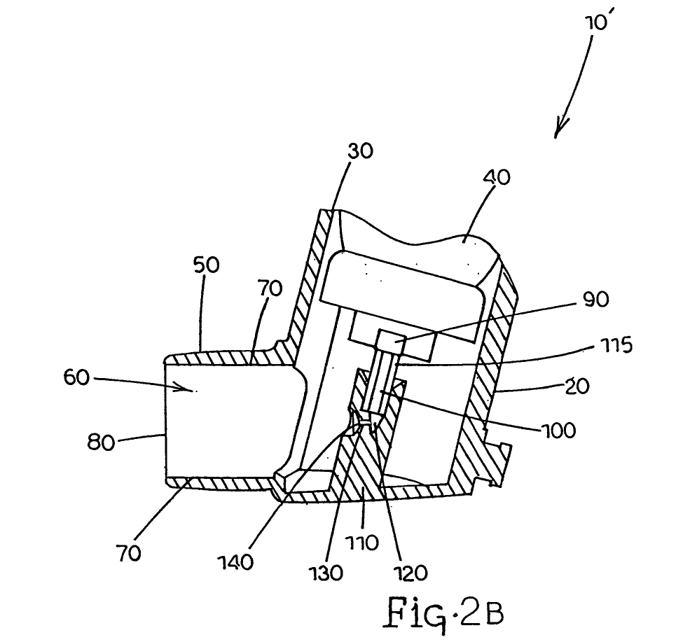

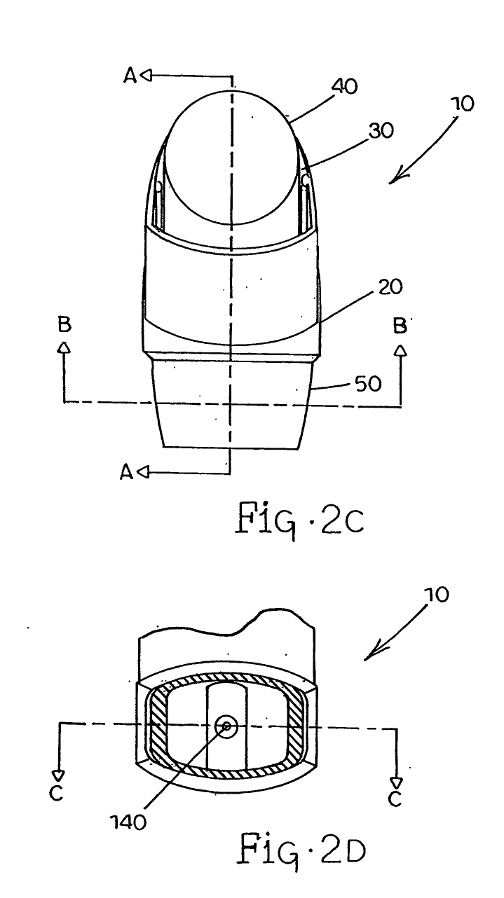

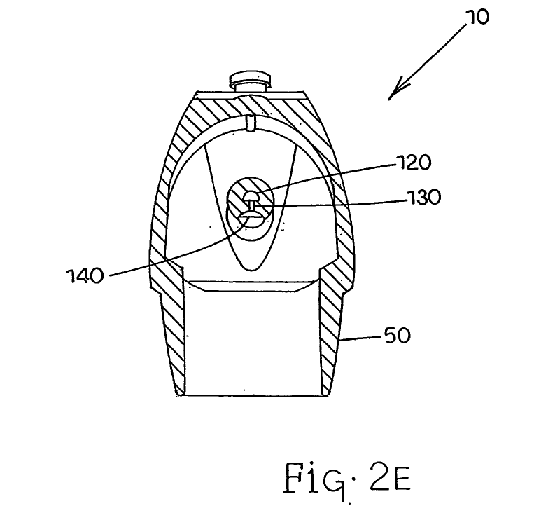

FIGS. 2A through 2E respectively illustrate perspective, side cross-sectional, top, frontal and bottom cross-sectional views of a conventional oral inhaler 10.

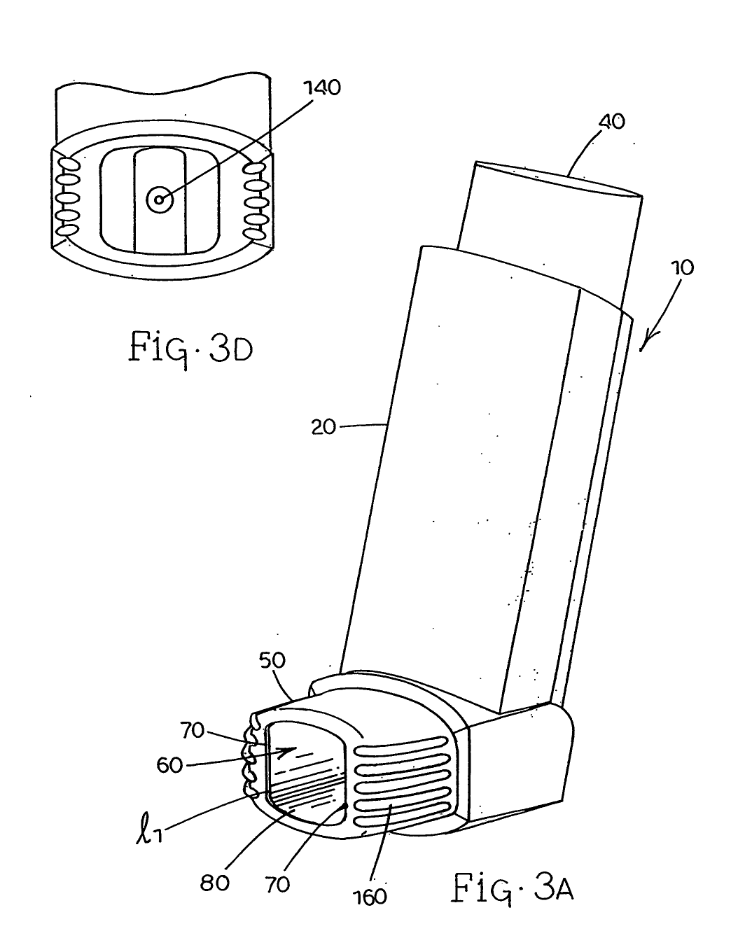

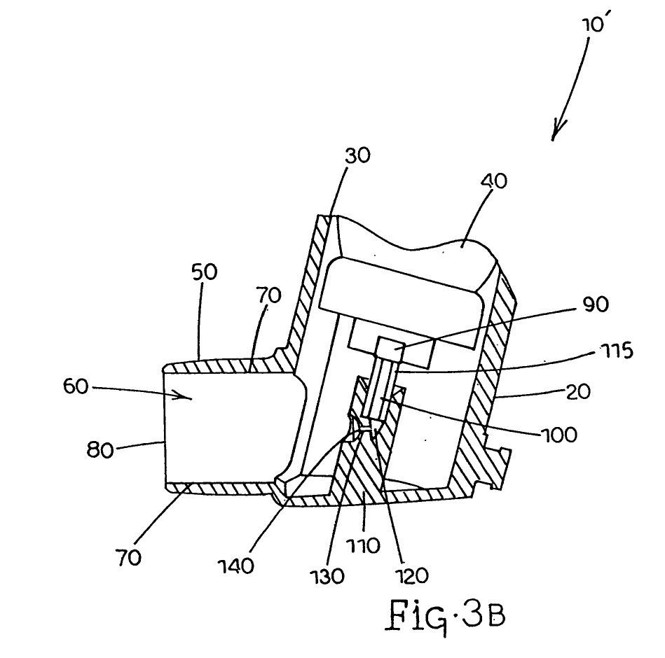

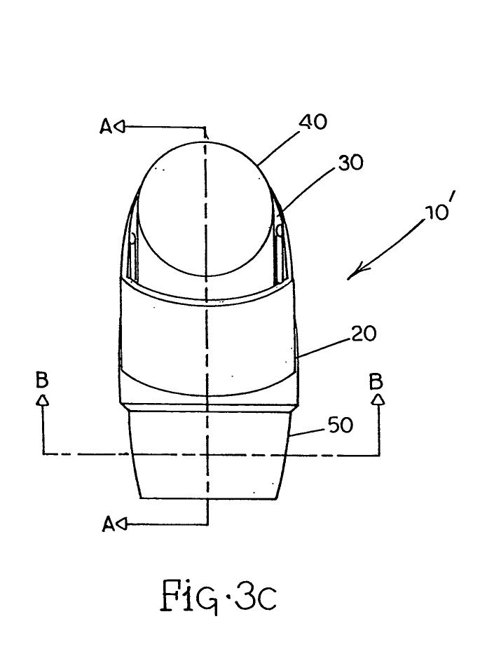

FIGS. 3A through 3D illustrate various views of an embodiment of an oral inhaler 10′ in accordance with the present invention. As shown in FIGS. 3A and 3D, a plurality of protrusions 160 are present on opposing sides of the mouthpiece and extend coaxially along the axis 11.

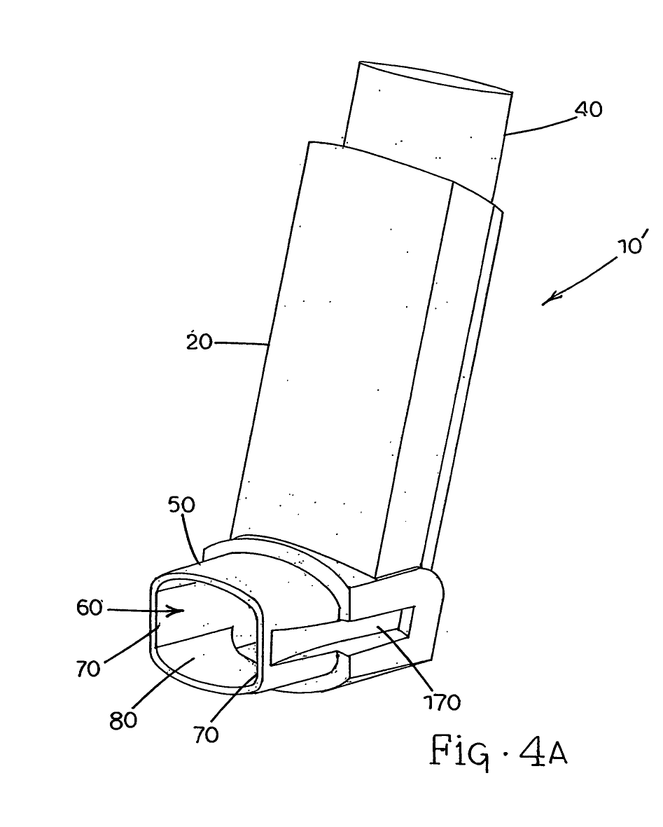

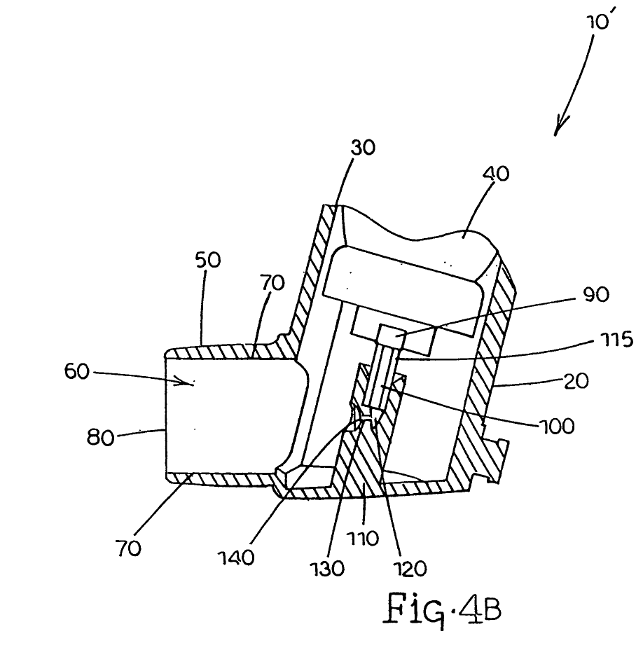

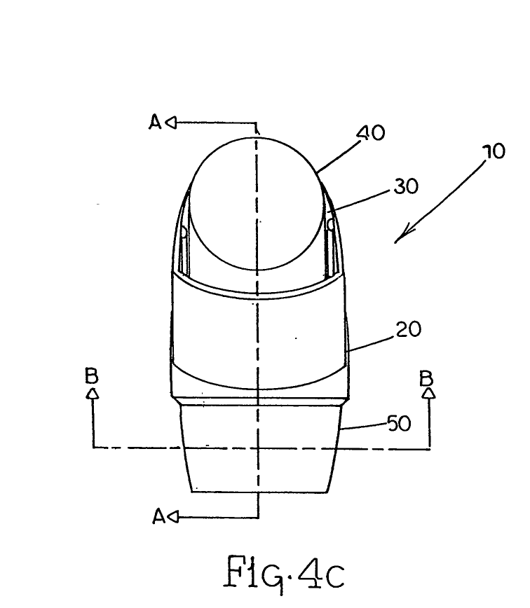

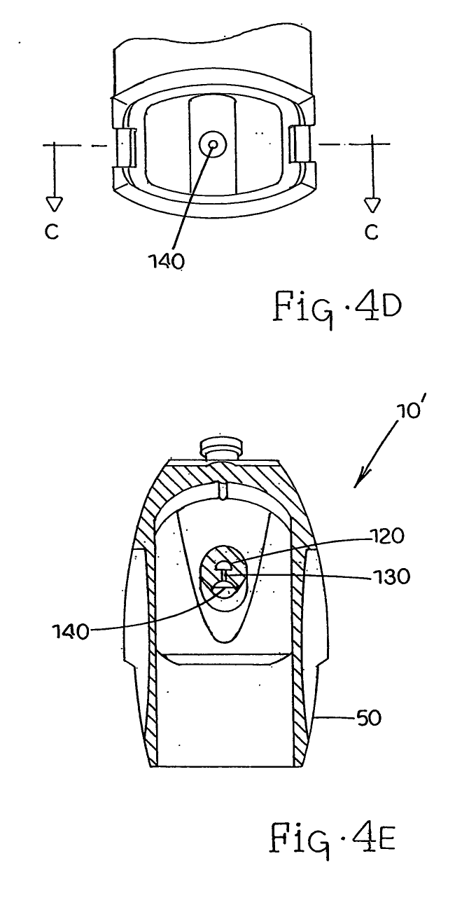

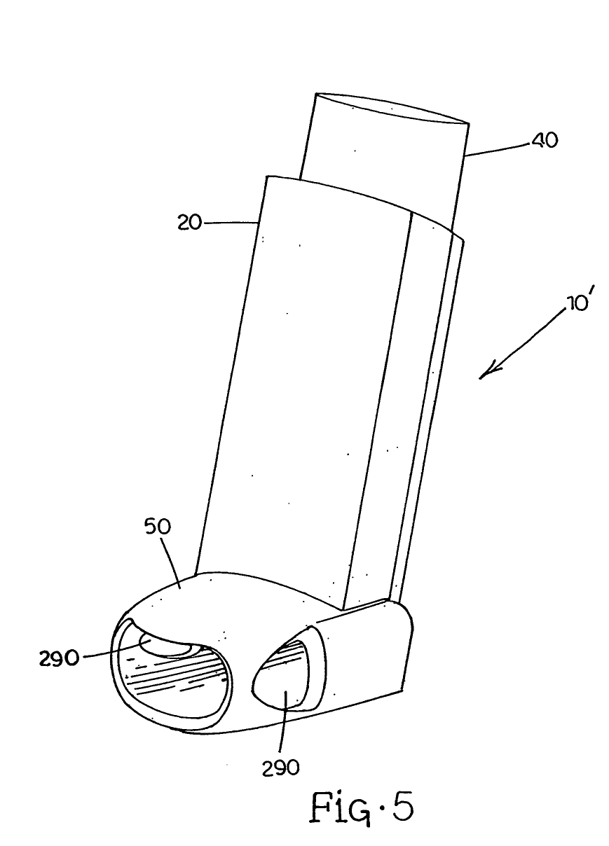

FIGS. 4A through 4E depict an additional embodiment of an oral inhaler 10′ in accordance with the invention. In this instance, indentations (denoted as 170) are present on opposing sides of the outer surface of mouthpiece 50. As shown, indentations 170 extend along with the longitudinal axis I₁ of the mouthpiece 50.

FIG. 5 illustrates a perspective view of another embodiment of an oral inhaler 10′ according to the present invention. Although not shown, the inhaler 10′ may have an internal assembly similar to that illustrated in FIGS. 1B, 2B, 3B, and 4B, although other configurations may also be employed. Referring to FIG. 5, two openings (denoted as 290) are present in opposing sides of mouthpiece 50 and extend along the longitudinal axis 11 of mouthpiece 50. Notwithstanding the embodiment illustrated in FIG. 5, it should be emphasized that other numbers of openings may be present in the mouthpiece, and in a different or similar configuration to that set forth in FIG. 5.

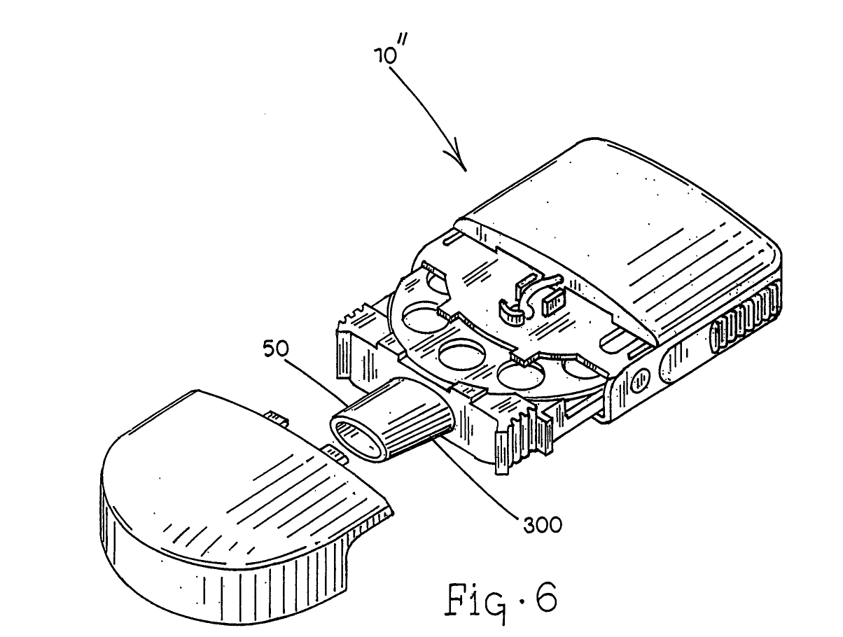

An embodiment of a DPI structured in accordance with the present invention is presented in FIG. 6 and is denoted as 10″. The DPI 10″ is described in U.S. Pat. No. 4,811,731. In summary, the inhaler 10″ includes four principal components, namely housing 300, tray 310, rotatable support 320, and cover 330. Extending from the front of the tray is mouthpiece 50 through which medicament exits the device as it is inhaled by the patient. As seen in FIG. 6, an opening 300 is present which extends along the length of the mouthpiece 50. Although not visible from this view, a second opening may be present on the opposite side of the mouthpiece 50.

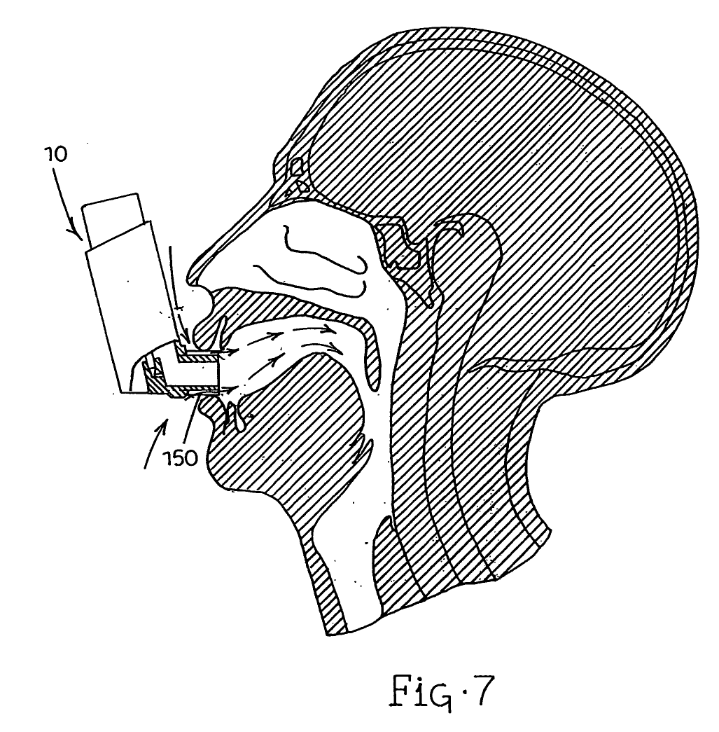

FIG. 7 illustrates a side view of a patient employing oral inhaler 10′ according to the present invention. More specifically, in this embodiment, a plurality of protrusions 150 are present on mouthpiece 50 similar to the embodiment illustrated in FIGS. 1A through 1D. In accordance with the invention, the presence of the protrusions 150 relative to the oral cavity of the patient allows for void spaces between the outer surface of the mouthpiece and the patient. Accordingly, and as represented by the arrows in FIG. 7, air flow channels through the void spaces are present to facilitate intake of medicament from the inhaler by the patient. Thus, the patient is advantageously allowed to inhale freely during dose delivery without an airflow path internal to the inhalation device or an additional mouthpiece component being required.

The invention has been described in detail with respect to the embodiments described hereinabove. However, it should be appreciated that such embodiments are set forth for illustrative purposes only, and are not used to limit the scope of the invention as defined by the claims.

That which is claimed:

1. An oral inhaler suitable for delivering a pharmaceutical formulation to a patient, said inhaler comprising:
a container having the pharmaceutical formulation comprising at least one medicament present therein; and
a mouthpiece configured for oral engagement with a patient and in communication with said container, the mouthpiece having an inner surface and an outer surface;
wherein the outer surface of the mouthpiece contains at least one longitudinally-extending disuniformity such that when the patient orally engages the mouthpiece at least one void space is created between the outer surface of the mouthpiece and the oral cavity of the patient so as to provide an air flow channel through the at least one void space to facilitate intake of the at least one medicament by the patient.

2. The inhaler according to claim 1, wherein the at least one longitudinally-extending disuniformity is selected from the group consisting of at least one protrusion, at least one indentation, at least one opening in the outer surface of the mouthpiece, and combinations thereof.

3. The inhaler according to claim 1, wherein said at least one longitudinally-extending disuniformity comprises a plurality of protrusions.

4. The inhaler according to claim 3, wherein the plurality of protrusions are equidistant from one another.

5. The inhaler according to claim 3, wherein the plurality of protrusions are present opposite to each other along respective sides of the mouthpiece.

6. The inhaler according to claim 3, wherein the protrusions are present throughout the outer surface of the mouthpiece.

7. The inhaler according to claim 3, wherein said at least one protrusion is present as one protrusion.

8. The inhaler according to claim 1, wherein the said at least one longitudinally-extending disuniformity comprises a plurality of indentations.

9. The inhaler according to claim 8, wherein the plurality of indentations comprise two indentations present opposite to each other along opposing sides of the mouthpiece.

10. The inhaler according to claim 1, wherein the longitudinally-extending disuniformity is present as at least one opening.

11. The inhaler according to claim 10, wherein the at least one opening comprises a plurality of openings.

12. The inhaler according to claim 11, wherein the plurality of openings comprise two openings each present opposite to each other on opposing sides of the mouthpiece.

13. The inhaler according to claim 1, wherein the pharmaceutical formulation comprises at least one medicament.

14. The inhaler according to claim 13, wherein the at least one medicament is selected from the group consisting of analgesics, anginal preparations, antiinfectives, antihistamines, anti-inflammatories, antitussives, diuretics, hormones, therapeutic proteins and peptides, and combinations thereof.

15. The inhaler according to claim 13, wherein the at least one medicament comprises albuterol sulphate.

16. The inhaler according to claim 13, wherein the at least one medicament comprises salmeterol xinafoate.

17. The inhaler according to claim 13, wherein the at least one medicament comprises fluticasone propionate.

18. The inhaler according to claim 13, wherein the at least one medicament comprises beclomethasone dipropionate.

19. The inhaler according to claim 13, wherein the at least one medicament comprises salmeterol xinafoate and fluticasone propionate.

20. The inhaler according to claim 1, wherein the inhaler is a metered dose inhaler.

21. The inhaler according to claim 20, wherein the pharmaceutical formulation is a pharmaceutical aerosol formulation comprising at least one medicament and at least one propellant.

22. The inhaler according to claim 21, wherein the at least one propellant is a C₁₋₄ hydrofluoroalkane.

23. The system according to claim 22, wherein the C₁₋₄ hydrofluoroalkane is selected from the group consisting of 1,1,1,2-tetrafluoroethane, 1,1,1,2,3,3,3-heptafluoropropane, and combinations thereof.

24. The inhaler according to claim 1, wherein the inhaler is a dry powder inhaler.

25. The inhaler according to claim 24, wherein the pharmaceutical formulation is a dry powder pharmaceutical formulation comprising at least one medicament and at least one ingredient.

26. The inhaler according to claim 25, wherein the at least one ingredient is selected from the group consisting of excipients, lubricants, binders, disintegrators, solvents, solubilizers, co-solvents, and mixtures of the above.

27. An oral inhaler system for delivering a pharmaceutical formulation to a patient, said inhaler comprising:
a container having the pharmaceutical formulation comprising at least one medicament present therein, the at least one medicament selected from the group consisting of analgesics, anginal preparations, antantiinfectives, antihistamines, anti-inflammatories, antitussives, diuretics, hormones, therapeutic proteins and peptides, and combinations thereof; and
a mouthpiece configured for oral engagement with a patient and in communication with said container, the mouthpiece having an inner surface and an outer surface;
wherein the outer surface of the mouthpiece contains at least one longitudinally-extending disuniformity such that when the patient orally engages the mouthpiece at least one void space is created between the outer surface of the mouthpiece and the patient so as to provide an air flow channel through the at least one void space to facilitate intake of the at least one medicament by the patient, wherein the at least one longitudinally-extending disuniformity is selected from the group consisting of at least one protrusion, at least one indentation, at least one opening in the outer surface of the mouthpiece, and combinations thereof.

28. A method of administering at least one medicament to a patient, said method comprising:
providing an inhaler as defined by claim 1; and
activating the inhaler to deliver the at least one medicament to the patient.

29. The method according to claim 28, wherein the at least one medicament is selected from the group consisting of analgesics, anginal preparations, antiallergics, antiinfectives, antihistimines, anti-inflammatories, antilussives, diuretics, hormones, therapeutic proteins, peptides, medicaments for treating erectile dysfunction, and combinations thereof.

30. The method according to claim 28, wherein the at least one medicament is selected from the group consisting of fluticasone, beclomethasone, salmeterol, albuterol, ipratropium, salts thereof, esters thereof, solvates thereof, and combinations thereof.

31. The method according to claim 28, wherein the at least one medicament comprises albuterol sulfate.

32. The method according to claim 28, wherein the at least one medicament comprises salmeterol xinafoate and fluticasone propionate.

33. The method according to claim 28, wherein the at least one medicament comprises fluticasone propionate.

34. The method according to claim 28, wherein the at least one medicament comprises beclomethasone dipropionate.
